(19) United States
(12) Patent Application Publication
(10) Pub. No.: US 2015/0314029 A1
(43) Pub. Date: Nov. 5, 2015

(54) Decontamination Device and Method

(71) Applicant: PHOCATOX TECHNOLOGIES, LLC, Zionsville, IN (US)

(72) Inventors: Harper Jones, Houston, TX (US); Mark Brodowicz, Charlotte, NC (US)

(21) Appl. No.: 14/702,093

(22) Filed: May 1, 2015

Related U.S. Application Data
(60) Provisional application No. 61/987,514, filed on May 2, 2014.

(51) Int. Cl. A61L 9/12; A61L 9/04; A61L 2/20

(52) U.S. Cl. CPC A61L 9/12; A61L 2/202; A61L 2/208; A61L 9/04; A61L 2/20

(57) ABSTRACT

The present invention relates to a device and method for decontamination. The device and method can be used to eradicate airborne and surface organic molecules and microorganisms, including pathogens, bacteria, and imbedded odors either on surfaces or items. The device includes a means to generate vapor-phase hydrogen peroxide, purified ozone and hydrogen radicals using UV light which treats fluids in multi-chambers, with passive or active mixing of the fluids taking place, prior to release of the treated fluid onto the surfaces or items to be treated. The method includes use of the device noted in an enclosed space, operated at a specified temperature and relative humidity range, by an operator outside the enclosed space. The surfaces and items are placed within the enclosed space for treatment.

### CROSS-REFERENCE TO RELATED APPLICATIONS

The benefit of U.S. Provisional Patent Application No. 61/987,514 (filed May 2, 2014) is claimed, and that provisional application is hereby incorporated by reference.

### STATEMENT REGARDING FEDERAL SPONSORED RESEARCH OR DEV

Not applicable.

### FIELD OF THE DISCLOSURE

The present disclosure relates to a decontamination device and method to be used in restoration and decontamination applications. Any number of issues can cause toxic or odor conditions on personal items, furniture or similar objects. The present disclosure could be used to eradicate airborne and surface organic molecules and microorganisms, including pathogens, bacteria, and imbedded odors. This disclosure uses a device and method with a design that incorporates multiple chambers and mixing, to generate increased production and subsequent effectiveness of vapor-phase hydrogen peroxide, purified ozone and hydrogen radical, which generates more effective results in a reduced amount of time.

### BACKGROUND OF THE INVENTION

Historically, the elimination of toxic conditions or odor on items has been addressed through a variety of means. These include technologies applicable to all manner of fluids, including air and liquids. One such method is the use of ultraviolet radiation or “UV.” UV can kill a broad range of harmful microorganisms and the most common technique is through the use of UV lamps. A related method is through the generation of ozone, which is then used to treat the fluid at issue. Such methods and apparatuses may be advantageously used to reduce or eliminate the need for chemicals, aerosols, preservatives, microfiltration, and like materials and processes for the sterilization of liquids and/or gases, however there is room for improvement from the prior art.

As for the prior art, an apparatus for irradiating contaminants by means of a UV light involving a single-pass through chamber, with no means for mixing, have been widely described, such as in Wedekamp (U.S. Pat. No. 4,948,980). Wedekamp provides an apparatus consisting of a single chamber pass-through tubular body through which the medium to be irradiated flows, and at least two UV light sources with reflectors arranged externally to the tubular body and having parallel axes. Similarly, Clark (U.S. Pat. No. 7,875,247) describes a UV air sterilization device consisting of a single chamber comprising inner surfaces having a diffuse reflective behavior. The sterilization chamber includes an inlet aperture and an outlet aperture for air to flow through the chamber and a light source emitting a UV light, but lacks any means for mixing, either passive or active. Further, Morrow (U.S. Pat. No. 6,589,489) discloses an air purifier with a single chamber with an air flow path with a dielectric body interposed across the path with a source of UV upstream from the body. Likewise, continuous wave UV light (CWUV) has been proposed for sanitation of air in HVAC systems. For example, in Matschke (U.S. Pat. No. 6,022,511) a sterilization system is disclosed that replaces one or more sections of air ducts with a single-chamber device of ellipsoidal ducts containing ultraviolet light sources and having parabolic or ellipsoidal reflectors disposed in an inlet and an outlet for flow of air.

Similarly, in U.S. Pat. No. 6,228,332 (Dunn), short-duration, high-intensity pulsed broad-spectrum polychromatic light is used to treat water in a single chamber, with no mixing, for the deactivation of microorganisms. As described in Dunn, deactivation involves illuminating the fluid with at least one short-duration, high-intensity pulse of broad-spectrum polychromatic light. The system includes a single chamber housing having an inlet port and an outlet port for the fluid flow. A tubular light source for deactivating microorganisms and a tubular baffle for directing the fluid flow are positioned within the housing. Fluid enters the inlet port and flows between the housing and the tubular baffle in one direction, around the end of the tubular baffle and back through the center of the tubular baffle in a second direction exiting the outlet port. In this case, fluid flows around the tubular light source, which provides at least one short-duration, high-intensity pulse of broad-spectrum polychromatic light. However, not described in Dunn is the use of a multi-chamber systems nor one that involves any mixing.

Another common method for treating fluids is through the creation of ozone. As seen in the single chamber device in Andrews (U.S. Pat. No. 4,882,129) and others. The downside with the use of high levels of ozone when using corona discharge production methods to treat items in air, is the corrosive nature of the ozone on any of the items treated, including on the generating equipment itself. This has potential long-term localized environmental impacts.

While the prior art treats the fluids at issue using a single-chamber structure with no means for passive or active mixing, whereby the fluid flows through the structure and is treated in such a manner that the resulting fluid is, admittedly, cleaner than if it had not been treated at all, but not as clean as the present disclosure. The present disclosure uses a device which includes multiple chambers and a mixing means, passive or active, which creates additional fluid mixture, to a create fluid which is of such cleaning intensity, that it actually becomes a cleaning instrument in itself. This device can be coupled with a novel method in order to achieve more favorable results, in a shorter time, using less resources. An additional benefit is that the disclosed method and device are environmentally friendly and leave no toxic residue or offending odor.

### BRIEF SUMMARY OF THE INVENTION

To this end, the subject of the invention is a device and method for decontaminating fluids and then using the treated fluids to treat toxic or odor conditions on surfaces and items, including airborne and surface organic molecules and microorganisms. This devices includes generating vapor-phase hydrogen peroxide, purified ozone and hydrogen radicals using known methods but within a device that incorporates multiple chambers and a means for mixing the fluid contents of the chambers, either passively or actively, before the treated fluid can then be released into a enclosed area which contains the surfaces or items to be treated.

### BRIEF DESCRIPTION OF THE DRAWINGS

FIG. 1, is a schematic side view of a device made in accordance with an embodiment of this invention.

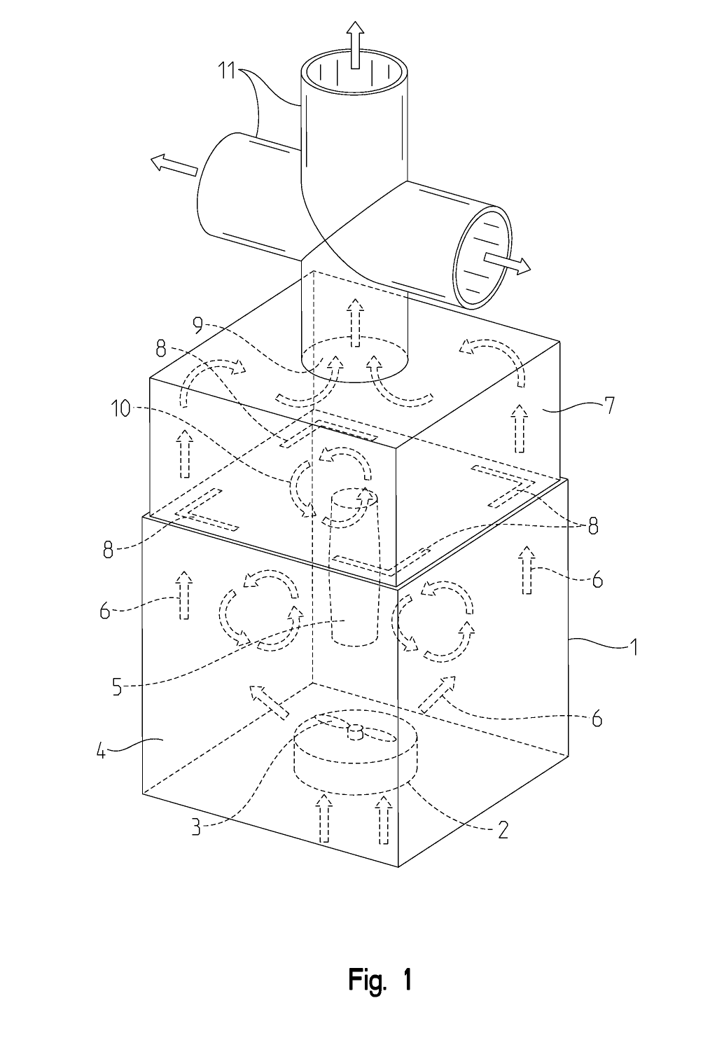

### DESCRIPTION

For the purpose of promoting an understanding of the principles of the present invention, reference will now be made to the embodiment illustrated in specific language contained herein. It will, nevertheless, be understood that no limitation of the scope of the invention is thereby intended; any alterations and further modifications of the described or illustrated embodiments and any further applications of the principles of the invention as illustrated therein are contemplated as would normally occur to one skilled in the art to which the invention relates.

The preferred embodiment of the device in this disclosure would be a square-shaped housing 1 with an intake 2 for the fluid to enter the unit. This intake would include a mechanical means to bring the fluid into the unit and ultimately move the fluid through the unit, such as a small fan 3. Inside the first chamber 4 of the unit would be a means 5 to generate vapor-phase hydrogen peroxide, purified ozone and hydrogen radicals using UV light (“fluid agents”) 6, from commonly known methods. Due to the fact that the first chamber is not completely open to the second chamber 7, passive mixing of the fluid and fluid agents would take place in the first chamber, prior to the fluid and fluid agents passing to the second chamber. The first chamber 4, would be fixedly connected to the second chamber 7, which is in the same shape as the first chamber. The openings 8 between the first and second chamber, in relation to the first chamber, would be on the opposite side from the intake 2. In the preferred embodiment these openings 8 would consist of L shaped slits located near the angular corners of the first chamber 4. The placement and shape of the openings are such to encourage mixing of the fluid and fluid agents.

The fluid and fluid agents would pass through these openings 8 and enter the second chamber 7. The second chamber would be dimensionally similar to the first chamber. Due to the location of the exit 9 being in a position that does not allow direct, straight and unimpeded movement of the fluid and fluid agents from the openings to the exit, additional passive mixing is seen 10, resulting in a treated fluid mix. The exit for the treated fluid 9 would be located on the opposite side from the openings 8 and would be circular in shape and located in the center of that side of the second chamber. From the exit 9, the treated fluid would travel into a duct system 11, which would provide uniform distribution of the treated fluid in several directions.

Due to the shape of the device and the treatment of the fluids therein, the amount and intensity of the fluid agents and thus the treated fluids, would be better optimized than seen in the prior art. This comes as a result of the multi-chamber system as well as mixing (passive or active) which allows for placement of more fluid agents in a smaller space. This increase in mixing arises as a result of the placement and shape of openings 8, between the first and second chambers as well as the placement of the exit 9. The treated fluid then leaves the device via the exit 9 and enters a means to direct the treated fluid in selected directions 11, such as multi-port tubing or similar means. Upon their exit from the device, the treated fluid would then come into contact with the item or items to be treated. The amount of time necessary for the treatment of the surface or item would depend upon the type of result desired, but these treatment times are going to be significantly less than those seen through use of the prior art.

Testing conducted by the applicants support this assertion:

| | O3 PPM |
|---|---|
| Unit with single chamber | 40.66 |
| Unit with two chambers | 60.33 |
| (preferred embodiment) | |

[Tests conducted at 64° F. and 56% RH. Measurement of 03 is one measure of total fluid agents present, which directly corresponds to the effectiveness of treatment].

While the preferred embodiment would be used for the treatment of air, it is anticipated that any fluid could be treated with this unit.

Another embodiment of the unit could incorporate an active means for mixing the fluid and fluid agents in chambers 1 and 2, such as impellers or small fans.

Likewise a further embodiment of the unit could eliminate the duct system or multi-port tubing and simply have the treated fluid leave the unit at the exit 9.

A further embodiment could incorporate the use of 3 or more chambers and could include the use of one or more means to generate vapor-phase hydrogen peroxide, purified ozone and hydrogen radicals using UV light (“fluid agents”) in a multitude of chambers.

A further embodiment could be a unit which is of a cylindrical shape but contains multiple chambers and openings. Between the chambers of this embodiment a number of slits or openings would be cut between the chambers, approximately mirroring the curvature of the cylinder, and located near the outside edge of the cylinder. In this way, mixing of the fluid and fluid agents would be facilitated as the fluid and fluid agents pass from one chamber to the next.

The effectiveness of the unit can be increased through the use of a specific method. In this method, the device is placed or mounted into an enclosed area whereby the treated fluid which is generated by the device can be contained within the enclosed area. For this method, the optimum ambient temperature for the enclosed area, but outside the device, is in the 70-85 degree Fahrenheit range, however the device can effectively operate in the range from 35 to 69 degrees Fahrenheit, well as from 86 to 110 degrees Fahrenheit. The optimum ambient relative humidity of the enclosed area should be equal to, or greater than, 50%. Additionally, under this method, for the ease and safety of the operator, there would be means to operate the device while the operator is outside the enclosed area.

While the invention has been illustrated and described in detail with the foregoing description, the same is to be considered as illustrative and not restrictive in character, it being understood that only the preferred embodiment has been described and that all changes and modifications that come within the spirit of the invention are desired to be protected.

What is claimed is:

1) A DEVICE for decontamination, comprising:
a) a first enclosed chamber 1 with an opening 2 for the purpose of bringing fluid into the chamber using a fan 3;
b) at least one source of ultraviolet radiation 5 within the first enclosed chamber;
c) openings 8 located in the first enclosed chamber which will allow fluid passage from the first enclosed chamber;
d) a second enclosed chamber 7 fixedly attached to the first enclosed chamber and sharing the side of the first enclosed which contains the openings 8, whereby fluid can move from the first enclosed chamber to the second enclosed chamber;
e) an opening 9 in the second enclosed chamber which is on the opposite side of the side in which the openings 8 are located, but not located directly across from them, whereby the fluid contained in the second enclosed chamber can leave the chamber;
f) an attachment 11 fixedly attached to the exit 9 located in the second enclosed chamber, which would allow the fluid to be directed in several distinct directions.

2) The DEVICE of claim 1 further comprising mechanical blades located in the second enclosed chamber for the purpose of mixing the fluid contained therein.

2) The DEVICE of claim 1 further comprising mechanical blades located in the first enclosed chamber for the purpose of mixing the fluid contained therein.

3) The DEVICE of claim 1 further comprising mechanical blades located in the first and second enclosed chambers for the purpose of mixing the fluid contained therein.

4) The DEVICE of claim 1 whereby the fluid is ambient air.

5) The DEVICE of claim 1 whereby the shape of the device is a square or rectangle.

6) The DEVICE of claim 1 whereby there is no attachment 11, allowing the fluid contained in the second enclosed chamber to exit via the opening 9.

7) The DEVICE of claim 1 whereby the shape of the device is cylindrical.

8) The DEVICE of claim 1 whereby the openings 8 are located in the corners of the chambers, if angular, or near the outside edge, if the device is cylindrical.

9) The DEVICE of claim 1 whereby the attachment 11 has multi-port tubing.

10) The DEVICE of claim 1 whereby the opening 9 is circular in shape.

11) A DEVICE for decontamination, comprising:
a) three or more enclosed chambers with an opening in the first chamber for the purpose of bringing fluid into the first chamber using a fan;
b) at least one source of ultraviolet radiation within the first enclosed chamber;
c) openings located in the first enclosed chamber which will allow fluid passage from the first enclosed chamber to one or more of the other chambers;
d) two or more enclosed chambers fixedly attached to the first enclosed chamber and sharing the side of the first enclosed which contains the openings, whereby fluid can move from the first enclosed chamber to two more enclosed chambers attached;
e) an opening in the two or more enclosed chambers which is on the opposite side of the side in which the openings are located, but not located directly across from them, whereby the fluid contained in the two or more enclosed chambers can leave the chambers.

12) The DEVICE of claim 11 further comprising mechanical blades located in the two or more enclosed chambers for the purpose of mixing the fluid contained therein.

13) The DEVICE of claim 11 further comprising mechanical blades located in the first enclosed chamber for the purpose of mixing the fluid contained therein.

14) The DEVICE of claim 11 further comprising mechanical blades located in the all enclosed chambers for the purpose of mixing the fluid contained therein.

15) The DEVICE of claim 11 whereby the shape of the device is a square or rectangle.

16) The DEVICE of claim 11 whereby the shape of the device is cylindrical.

17) The DEVICE of claim 11 whereby the openings are located in the corners of the chambers, if angular, or near the outside edge, if the device is cylindrical.

18) A METHOD of decontamination using a Device according to claim 1 comprising:
(a) a chamber of sufficient size to fit the Device according to claim 1 and at least one item to be treated, which chamber has the ability to be closed in an approximately air-tight manner;
(b) a switch in which to turn on and turn off the Device from outside the chamber when it is closed, while the Device would be inside the enclosed chamber;
(c) the temperature inside the enclosed chamber, but outside the Device, being between 35 and 110 degrees Fahrenheit;
(d) the relative humidity inside the enclosed chamber being equal to or greater than 50%;
(e) the placement of at least one item to be treated inside the enclosed the chamber;
(f) turning on the Device for a period of time, said time dependent upon the measure of cleaning sought for the item.

19. A METHOD of decontamination using a Device according to claim 11 comprising:
(a) a chamber of sufficient size to fit the Device according to claim 11 and at least one item to be treated, which chamber has the ability to be closed in an approximately air-tight manner;
(b) a switch in which to turn on and turn off the Device from outside the chamber when it is closed, while the Device would be inside the enclosed chamber;
(c) the temperature inside the enclosed chamber, but outside the Device, being between 35 and 110 degrees Fahrenheit;
(d) the relative humidity inside the enclosed chamber being equal to or greater than 50%;
(e) the placement of at least one item to be treated inside the enclosed the chamber;
(f) turning on the Device for a period of time, said time dependent upon the measure of cleaning sought for the item.
